(19) United States
(12) Patent Application Publication
(10) Pub. No.: US 2013/0183734 A1
(43) Pub. Date: Jul. 18, 2013

(54) Phospholipase-Carrier Complex

(71) Applicant: Clariant Produkte (Deutschland) GmbH, Frankfurt am Main (DE)

(72) Inventors: Kirstin Suck, Munich (DE); Friedrich Ruf, Tiefenbach-Ast (DE); Ulrich Sohling, Freising (DE)

(73) Assignee: CLARIANT PRODUKTE (DEUTSCHLAND) GMBH, Frankfurt am Main (DE)

(21) Appl. No.: 13/730,239

(22) Filed: Dec. 28, 2012

Related U.S. Application Data
(63) Continuation of application No. PCT/EP2011/061135, filed on Jul. 1, 2011.

(30) Foreign Application Priority Data
Jul. 1, 2010 (DE) 10 2010 025 764.8

(51) Int. Cl. C12N 11/14

(52) U.S. Cl. CPC C12N 11/14
USPC 435/180; 435/176; 435/177; 435/271

(57) ABSTRACT

A phospholipase-phospholipase-carrier complex for degumming crude oils. The phospholipase-carrier complex includes at least one phospholipase A1, A2 or B and at least one carrier, such as silicic acid, precipitated silicic acid, polyacrylate, polymethacrylate or divinylbenzene-crosslinked methacrylate. The disclosure further relates to a method of degumming crude oils and to a method of producing the phospholipase-carrier complex.

### CROSS REFERENCE TO RELATED APPLICATIONS

This application is a continuation application of international patent application PCT/EP2011/061135, filed Jul. 1, 2011, designating the United States and claiming priority from German application 10 2010 025 764.8, filed Jul. 1, 2010, and the entire content of both applications is incorporated herein by reference.

### FIELD OF THE INVENTION

The invention relates to the use of a phospholipase-carrier complex for degumming crude oils. Furthermore, the invention relates to a method of degumming crude oils and a phospholipase-carrier complex.

### BACKGROUND OF THE INVENTION

Crude oils contain—depending on the nature of the oil—undesirable minor constituents, which must be removed. To do this, the crude oils are refined. Refining improves the quality and stability of the oils. For this, various substances, including free fatty acids, metal ions, flavoring materials as well as phospholipids must be removed from the crude oil. For example, biodiesel is a product that is obtained by transesterification of vegetable oil with methanol. The contents of certain components, such as phosphorus or metal ions, in the biodiesel final product are limited according to the specifications of the EU and of the US standard (see EU standard specification for biodiesel EN 14214. In this case, for example, the upper limit for phosphorus is 4 ppm). As chemical processing of vegetable oils will be carried out increasingly in the future, the refining of these is particularly important.

Phosphorus impurities can poison catalysts, e.g. in hydrogenation reactions.

Crude oils contain so-called water-soluble and water-insoluble phospholipids. Water-soluble phospholipids can be extracted from the oils by hydration. The water-insoluble phospholipids remain in the oil and can for their part be removed for example with enzymes, such as phospholipases.

Phospholipases are enzymes belonging to the class of hydrolases, which hydrolyze the ester bond of phospholipids. Phospholipases are divided into 5 groups according to their regioselectivity with phospholipids:

Phospholipases A₁ (PLA₁), which cleave the fatty acid in the sn1 position with formation of 2-lysophospholipid.

Phospholipases A₂ (PLA₂), which cleave the fatty acid in the sn2 position with formation of 1-lysophospholipid.

Phospholipases C (PLC), which cleave a phosphoric acid monoester.

Phospholipases D (PLD), which cleave or exchange the head group.

Phospholipases B (PLB), which cleave the fatty acid both in the sn1 position and in the sn2 position with formation of a 1,2-lysphospholipid.

These reactions always take place at the interface of aggregated substrates.

There are numerous descriptions of the use of phospholipases, mainly phospholipase A, for degumming crude oils. One example is Lurgi's Enzymax® process EP 0513709 B1, in which the water-insoluble phospholipids are removed from a previously degummed edible oil using phospholipases (A2, A1, B).

DE 4339556 C1 describes, as further variant of this process, the reuse of the enzyme, in which it is dissolved out of a spent, sludge-containing aqueous phase by adding surfactants or solubilizers and is used again as a largely sludge-free solution, which contains at least 10% of the enzyme used.

As the production costs of enzymes are high, it must be possible to use them repeatedly. It must therefore be possible to separate them economically from the reaction mixture. One possibility is to bind the enzymes in a suitable way with a carrier. There are essentially two methods available for this: immobilization and encapsulation.

In the case of immobilization, the enzymes are bound to a carrier. Bonding can take place in various ways. Physical bonding can be achieved by adsorption of the enzyme to the surface of the carrier. The bonding takes place by hydrophobic interactions or by ionic forces, wherein charged groups of the enzyme interact with oppositely charged groups on the surface of the carrier. This method has the advantages of simple implementation as well as relatively little effect on the activity of the enzyme. However, there is the drawback that the enzymes can be displaced again relatively easily from the surface of the carrier. Irreversible bonding of the enzyme can be achieved through formation of a covalent bond between enzyme and carrier. In this case, however, it has to be accepted that in some circumstances the activity of the enzyme is decreased, as the enzyme can, for example, be fixed on the surface in such a way that the active center is no longer accessible. Finally, the stability of the enzyme-carrier complex can be further increased by crosslinking the enzymes with at least bifunctional molecules. This results in larger aggregates, which are less soluble. In this method, however, it is very difficult to control the immobilization. Furthermore, mostly a marked deactivation of the enzyme has to be accepted, as its conformation is altered considerably or the active center is no longer freely accessible.

In the second method the enzyme is enclosed in a spherical or tubular matrix. The matrix must be permeable to the educts and products of the catalyzed reaction, but not to the enzymes. Natural polymers, e.g. alginates, gelatin or agar, or also synthetic polymers, such as polyacrylamide or polyvinyl alcohol, are used for this technique. In this method, in reactions in organic media the enzyme is protected against inactivation by the solvent. As a drawback, however, it has to be accepted that the matrix can act as diffusion barrier for the educts and products of the enzyme-catalyzed reaction.

The methods of immobilizing enzymes are familiar to a person skilled in the art and are described in detail in the following reference: L. Cao (2005): Carrier-bound Immobilized Enzymes; Wiley-VCH Verlag Weinheim, Germany.

The immobilization of phospholipase A1 in sodium alginate particles or sodium alginate-chitosan particles is described in CN 100535094 C and in CN 101485366 A.

WO 2005086900 A2, p. 142-144, lists methods of immobilising phospholipases on carriers such as Sepharose, gelatin, glutaraldehyde, albumin-glutaraldehyde, chitosan-xanthan, alginates and agarose.

The immobilization of lecitase in gelatin hydrogel with subsequent crosslinking by glutaraldehyde is reported by Sheelu et al., J Am Oil Chem Soc (2008) 85:739-748. The immobilized enzyme is used for oil degumming in a so-called “spinning basket” bioreactor for degumming rice germ oil. In the hydrogel, the enzyme remains active in the reactor for 6 cycles, in contrast to the comparative adsorptive immobilization on Eupergit, Celite and XAD-7. On these carriers, the enzyme was no longer active after the 2nd cycle.

Furthermore, immobilization of phospholipases by covalent and adsorptive techniques is described in the following references: Fernandez-Lorente et al., Journal of Molecular Catalysis B: Enzymatic (2007) 47: 99-104; Bornscheuer et al., Enzymes in Lipid Modification (2005), Chapter 12; 217-291; Garcia et al., Grasas y aceites (2008) 59; 368-374; Nam and Walsh, Journal of Food Biochemistry (2005): 2.9:1-12; Kim et al., Enzyme and Microbial Technology (2001) 29:587-592; Chen et al., Journal of Molecular Catalysis B: Enzymatic (1998) 5: 483-490. The enzymes in immobilized form were characterized with respect to pH-stability, temperature stability, kinetics of the enzyme reaction etc. In one example, phospholipase 1 was immobilized covalently and chiral molecules were then produced.

Overall, to date, no method has been described in which a phospholipase enzyme can be used in several recycling steps in the degumming of crude oils, in order to make the process of enzyme-catalyzed oil degumming more cost-effective.

### SUMMARY OF THE INVENTION

The problem to be solved by the invention was therefore to find a possible way of using phospholipase in industrial processes such as the degumming of crude oils, wherein it has high activity and a long half-life and therefore can be used repeatedly.

Now, it was found, surprisingly, chat phospholipases A1, A2 and/or B or mixtures thereof can be immobilized particularly well on carriers and these phospholipase-carrier complexes according to the invention can be used surprisingly effectively in the degumming of crude oils.

The problem according to the invention was solved by using a phospholipase-carrier complex with the features as disclosed herein. In a first aspect the present invention therefore relates to the use of a phospholipase-carrier complex comprising at least one phospholipase A1, A2 and/or B and at least one carrier for degumming crude oils.

In a first preferred embodiment the carrier is selected from inorganic carriers such as silicates and organic polymers and copolymers.

In a second preferred embodiment the silicate is selected from the group of natural and synthetic silicates and layered silicates and mixtures thereof.

In another preferred embodiment the silicate is a synthetic silicate based on silicon dioxide, wherein silicic acid and precipitated silicic acid are especially preferred.

In another preferred embodiment the silicate is hydrophobic or partially hydrophobic. The hydrophobicity is preferably adjusted by treating the hydrophilic silicates with functionalized silanes or siloxanes, bearing reactive groups that can react with the silica particles with formation of Si—O—Si bonds. In this way the silica particles are modified covalently on their surface with alkyl groups. As a rule chlorosilanes, preferably trichlorosilanes, alkoxysilanes, of these preferably trialkoxysilanes, or silazanes are used for this. A frequently used silazane is for example hexamethyldisilazane. The alkyl groups of the silanes, with which the silica surface was modified, can optionally carry other functional groups, e.g. amino groups, phenyl groups or polymerizable double bonds.

Silicic acids can be produced thermally or by wet chemical methods, as described for example in K. H. Büchel et al, (1999): Industrielle Anorganische Chemie [Industrial inorganic chemistry]; Wiley-VCH Verlag Weinheim, Germany. Of the thermal methods, flame hydrolysis is the dominant process, in which tetrachlorosilane is decomposed in an oxyhydrogen flame. The resultant fumed silicic acid is X-ray amorphous and non-porous. In the case of wet chemical methods, the precipitation technique is the most important in the production of silicic acids. For forming silicic acid, in the precipitation process water is put in large stirred vessels and then water glass and acid, as a rule sulfuric acid, are added simultaneously. There is formation of colloidal primary particles, which agglomerate as reaction continues, and finally coalesce into aggregates. In contrast to the fumed silicic acids, precipitated silicic acids are generally mesoporous.

The silicate is also preferably a layered silicate, which in an especially preferred embodiment is an acid-activated layered silicate, in which the particles are bound together by a binder. Preferred methods of acid activation of layered silicates are described for example in DE 4405878 A1 and DB 4405876 A1. According to another preferred embodiment the acid-activated layered silicate is obtained by coating a layered silicate, preferably obtained from a natural source, or also synthetic, with an acid. These acid-activated layered silicates are also known as surface-modified bleaching earths, e.g. from F. Bergaya et al. (2006): Handbook of Clay Science; Elsevier Verlag Heidelberg, Germany. Preferred layered silicates according to the present invention are two-layer silicates, e.g. serpentine-kaolins as well as three-layer silicates, such as talc-pyrophilites, smectites, which include among others montmorillonite, beidelite, nontronite, saponite, hectorite or stevensite, vermiculites and micaceous clays as well as mixtures thereof.

The silicate can moreover contain at least one metal oxide selected from the group consisting of oxides of aluminium, calcium, magnesium, zinc, titanium, zirconium and mixtures thereof.

Especially preferred carrier materials are the products Sipernat®, especially Sipernat® D5, D10, D22 or D90 and Aerosils from the company Evonik, and CAB-O-SIL®, especially CAB-O-SIL® M-5 from the company Cabot and K-carriers from the company Süd-Chemie AG.

Carriers comprising at least one organic polymer and/or copolymer are also preferred in the context of the present invention.

Organic polymers and copolymers that are preferred in the context of the present invention are selected from the group consisting of polyacrylate, polymethacrylate, polymethylmethacrylate, polyethylene, polyethylene terephthalate, polytetrafluorethylene, polypropylene, polyvinyl styrene, polystyrene, styrene-divinylbenzene copolymers, polyamide and mixtures thereof, wherein divinylbenzene-crosslinked polymers, polymethacrylate, polyacrylate and polyvinyl styrene are especially preferred. The products Duolite® or Amberlite® from the company Rohm and Haas can be used as carriers. The products Lewatit®, especially Lewatit VP OC 1600, from the company Lanxess, a macroporous, divinylbenzene-crosslinked polymer based on methacrylate, are also especially preferred.

Carriers in the form of a powder or granules are basically preferred according to the present invention.

If carriers in the form of a powder are selected, the particle size of the powder is preferably adjusted so that the carrier can be separated from the reaction mixture without difficulty with a suitable method, for example filtration or centrifugation, within a suitable length of time.

Preferably a powder is used with an average particle size, measured according to Malvern in air, of from 0.1 to 250 μm, more preferably of 1-150 μm, especially preferably with a particle size of 5-100 μm, quite especially preferably with a particle size of 8-80 μm. Furthermore, the carrier can be used as granules, which have an average particle size, measured according to Malvern in air of more than 0.1 mm. Preferably the granules have a grain size in the range of from 0.15 to 5 mm, especially preferably 0.2 to 2 mm. The grain size can be adjusted for example by sieving from granules with wide particle size distribution, a method that is familiar to a person skilled in the art.

The size of the organic-based granules can be adjusted in the polymerization reaction. Non-limiting examples of polymerization techniques are emulsion polymerization, suspension polymerization, precipitation polymerization, solution polymerization and spray-polymerization. The desired particle size distribution can be adjusted subsequently, e.g. by sieving.

The inorganic-based granules can be produced by usual methods, for example by treating the finely ground carrier material with a granulating agent, for example water, and then granulating it in a usual granulating device in a mechanically produced fluidized bed. However, other processes can also be used for producing the granules. Thus, the pulverulent carrier material can for example be formed into granules by compaction.

Extrusion of a plastic paste is also possible. The extrudate is then comminuted, for example by chopping the extruded strand into short cylindrical pieces, and then the formed pieces obtained are dried. As well as solid cylinders, it is also possible to produce e.g. hollow cylinders in this way.

After forming, the inorganic granules can also be heat treated, and for example can be sintered by heating. The stability of the granules can be increased in this way. For the heat treatment, the granules are preferably heated to a temperature above 300° C., according to another embodiment to a temperature above 400° C., and according to yet another embodiment to a temperature above 500° C. According to one embodiment the temperature is selected as below 1200° C., according to another embodiment below 1000° C.

In order to obtain stable inorganic granules, the heat treatment is preferably selected for a duration of at least 30 minutes, according to another embodiment for a duration of at least 60 minutes. According to an embodiment that is also preferred, the treatment time is selected as less than 5 hours.

In another embodiment there is a favorable effect on the enzyme activity if a high proportion of silicic acid is selected during production of the granules based on precipitated silicic acids. The granulation mixture can contain other layered silicates in addition to the silicic acid. These layered silicates can serve as binder and lead to a higher strength of the granules. Bentonites are preferably used as these layered silicates. It is possible to use both layered silicates in the alkali form, especially the sodium form, and those with alkaline-earth ions as exchangeable cations, especially calcium ions. Examples of layered silicates are bentonites, montmorillonites, natronites, saponites, hectorites, attapulgites, sepiolites or mixtures thereof. The proportion of these layered silicates is preferably selected in the range of from 0.1 to 50 wt %. In addition, for example granulating aids or pore-forming agents can be contained in the pulverulent granulation mixture. For example, without being restricted to these, the following can be used as binders; agar-agar, alginates, chitosans, pectins, gelatins, lupinene protein isolates or gluten. The binder can also be of inorganic nature. Water glasses, bentonites or silica sol are usually used as inorganic binders. The percentages for the pulverulent granulation mixture refer to a dry, free-flowing granulation mixture, i.e. without addition of liquid.

Carriers that have a high BET specific surface are basically preferred in the context of the present invention, wherein a surface of above 10 m²/g is preferred, more preferably a surface of above 20 m²/g, especially preferably of above 30 m²/g, especially of above 40 m²/g, also preferably of above 50 m²/g and most preferably of above 50 m²/g. According to another preferred embodiment the specific surface is in the range of from 10 to 650 m²/g, especially preferably 30 to 520 m²/g, especially preferably 50 to 500 m²/g.

According to another preferred embodiment, the carriers used in the context of the present invention have a high pore volume. According to a preferred embodiment the carriers have a pore volume of more than 0.1 ml/g, especially preferably a pore volume of more than 0.2 ml/g, quite especially preferably a pore volume of more than 0.3 ml/g. The pore volume is determined as cumulative pore volume according to BJH (I. P. Barret, L. G. Joiner, P. P. Haienda, J. Am. Chem. Soc. 73, 1991, 373) for pores with a diameter of from 1.7 to 300 nm. According to one embodiment the carriers have a pore volume of less than 1.5 ml/g. According to another embodiment of the method the pore volume of the inorganic carrier material is less than 1.4 ml/g and according to another embodiment less than 1.3 ml/g. A pore volume of from 0.1 to 1.5 ml/g, especially of from 0.4 to 1.0 ml/g is especially preferred.

Carriers that have a minimum pore diameter of 2 nm are basically preferred in the context of the present invention. The pore diameter is determined by the BJH method. According to a preferred embodiment the pore diameter is more than 2 nm, especially preferably more than 5 nm, quite especially preferably more than 8 nm. A pore diameter of from 2 nm to 100 nm is especially preferred, especially of from 3 to 60 nm, more preferably of from 7 to 35 nm and most preferably of from 20 to 32 nm.

Carriers that have a pH measured in a 10% suspension in water of 2.0-9.0, preferably of 3.0-8.0, especially preferably of 3.0-7.5 are also preferred in the context of the present invention. Carriers with a pH of 2.0-9.0 have a favorable effect on the enzyme activity.

Furthermore, the present invention relates to a method of degumming crude oil, comprising the steps
a) mixing a phospholipase-carrier complex as defined above with a buffer solution in the ratio of 0.1%-80% (weight of phospholipase-carrier complex to volume of buffer solution;
b) contacting the buffer solution containing the phospholipase-carrier complex with a crude oil;
c) separating the crude oil from the buffer solution containing the phospholipase-carrier complex.

In another preferred embodiment, the method of degumming crude oil comprises the step
d) repeated contacting of the buffer solution containing the phospholipase-carrier complex with a crude oil.

In the context of the method according to the invention of degumming crude oil, the same buffer solutions are preferred as have already been defined above. The definitions given in the context of the method according to the invention of producing the phospholipase-carrier complex therefore also apply in the present case for the method according to the invention of degumming crude oil.

A preliminary degumming with acid at a temperature of from 25 to 95° C., preferably 35 to 85° C., is also possible.

In another preferred embodiment of the method according to the invention of degumming crude oil, the proportion of the buffer solution containing the phospholipase-carrier complex relative to the crude oil is adjusted to 0.01 to 30 wt %, preferably 0.05 to 20 wt %, more preferably to 0.1 to 15 wt % and especially preferably to 0.5 to 12 wt %, especially preferably to 1 to 10 wt % and most preferably of from 1 to 5 wt %.

More preferably the proportion of enzyme relative to the crude oil is adjusted to 0.01 to 20 units per gram of oil (U/g), more preferably 0.1 to 15 U/g, especially preferably 0.2 to 13 U/g. (units: international unit for enzyme activity; 1 unit corresponds to the substrate turnover of 1 μmol/min).

The mixing of the phospholipase-carrier complex with the buffer solution can take place in any way known by a person skilled in the art. In addition, mixing is possible in which the buffer solution is sprayed onto the phospholipase-carrier complex.

In another preferred embodiment, the contacting according to steps b) and/or d) is carried out for a period of from 1 minute to 24 hours, more preferably 5 minutes to 20 hours, more preferably of from 10 minutes to 18 hours and especially preferably of from 15 minutes to 10 hours and preferably for a period of from 20 minutes to 5 hours, especially preferably of from 25 minutes to 4 hours and most preferably for a period of from 30 minutes to 3 hours.

In an embodiment that is also preferred, the contacting according to steps b) and/or d) is carried out at a temperature of from 20 to 85° C., preferably 30 to 80° C., more preferably of from 32 to 75° C. and most preferably of from 35 to 65° C.

The contacting according to step b) and/or d) can take place by mixing methods of any kind, which are known as suitable by a person skilled in the art, such as shaking, stirring or ultrasound.

In the method according to the invention of degumming crude oil, any oil or fat derived from plants, animals, algae and fishes can be used. Non-limiting examples of preferred oils and fats are: soya oil, rape oil, palm oil, sunflower oil, canola oil, rice germ oil, peanut oil, coconut oil, pumpkin seed oil, maize germ oil, olive oil, jojoba oil, jatropha oil, walnut oil, grapeseed oil, sesame oil, almond oil, linseed oil or cottonseed oil. In the context of the present invention, mixtures of the oils or fats as well as mixtures of oils and fats of any kind can also be used.

According to a preferred embodiment, the crude oil is a previously degummed or previously conditioned crude oil (the two terms are used synonymously in the context of the present application). Previously degummed crude oil is obtained for example by mixing the oil with water, at a temperature between 30° C. to 90° C. for 15 to 60 minutes, preferably 30 to 60 minutes, wherein a temperature of from 35 to 85° C. is preferred and a temperature of from 40 to 80° C. is especially preferred. Moreover, previously degummed oil is obtained by treatment with acid, especially citric acid or phosphoric acid, at a temperature between 30° C. to 90° C. for 5 to 60 minutes, preferably 15 to 60 minutes, wherein a temperature of from 35 to 85° C. is preferred and a temperature of from 40 to 80° C. is especially preferred. In another possible embodiment the acid-containing aqueous phase is then separated e.g. by centrifugation. In a preferred embodiment, after the acid treatment, a neutralization step will take place with a corresponding base, in order to reach a pH of from 3.5 to 8.0, preferably of from 4 to 7. Then the oil can be separated from the gums obtained for example by centrifugation or filtration.

In another especially preferred embodiment the enzyme-carrier complex can be added directly to the previously conditioned neutralized oil and further processed.

In another preferred embodiment the oil is an untreated crude oil.

The solid phospholipase-carrier complexes produced by the method according to the invention are also suitable for continuous use, e.g. for use in the flow reactor or in a column packed with supported phospholipase, through which a solution of the substrate is then led continuously, as well as for use in processes carried out batchwise.

The buffer solution containing the phospholipase-carrier complex can be separated by any method that is known by a person skilled in the art to be suitable for the purpose according to the invention, and separation by centrifugation, filtration or settling is preferred.

In a preferred embodiment of the present invention, untreated, or previously degummed, or previously conditioned crude oil can be added again to the buffer solution containing the solid phospholipase-carrier complex. In another especially preferred embodiment, the (initial) concentration of the buffer solution is restored by topping-up the separated fraction of buffer solution and phospholipase-carrier complex with fresh buffer solution.

A particular advantage of the phospholipase-carrier complex according to the invention is that it can be reused several times, but at least three times, but even up to 250 times, preferably up to 200 times, more preferably up to 150 times and also preferably up to 100 times, especially preferably four to 30 times, especially 5 to 25 times, also preferably 6 to 20 times and most preferably 7 to 18 times.

With the method according to the invention it is possible to lower the phosphorus value in the degummed oil to below 20 ppm, especially preferably to below 10 ppm, quite especially preferably to below 4 ppm phosphorus.

Furthermore, with the method according to the invention it is possible to lower the calcium and magnesium content to below 20 ppm, especially preferably to below 15 ppm, quite especially preferably to below 10 ppm, also preferably to below 8 ppm and most preferably to below 4 ppm. In a quite especially preferred embodiment the calcium and magnesium content is lowered to below 3 ppm.

In another aspect the present invention relates to a method of producing a phospholipase-carrier complex according to the invention, comprising the steps:
a) providing at least one phospholipase A1, A2 and/or B or mixtures thereof in a buffer solution with a pH of from 3.0 to 8.0;
b) providing at least one carrier as defined above;
c) contacting the at least one phospholipase A1, A2 and/or B or mixtures thereof with the at least one carrier;
d) optionally separating the resultant phospholipase-carrier complex from the reaction medium.

The phospholipase is preferably provided in the form of an aqueous buffer solution preferably in citrate buffer with a pH of 5. In a preferred embodiment of the method according to the invention the concentration of the buffer solution is set in a range of from 5 to 1000 mmol/l, preferably in a range of from 10 to 500 mmol/l, more preferably 15 to 250 mmol/l and most preferably 30 to 150 mmol/l. Preferred buffers are acetate buffers and citrate buffers, wherein basically any buffer can be used that is known by a person skilled in the art to be suitable.

The pH of the buffer solution is selected depending on the enzyme to be immobilized and is preferably in the range of from 3.0 to 9.0, more preferably in the range of from 3.0 to 8.0 and most preferably in the range of from 3.0 to 7.0. A pH range of from 4.0 to 6.0 is also preferable. The ideal range for the pH depends on the specific enzyme. For phospholipases A1 and phospholipases A2 the pK of the buffer is preferably selected in the range of from 3.0 to 7.0.

In a preferred embodiment the concentration of the buffer is set in the range 10 to 300 mmol/l, more preferably 20 to 200 mmol/l and most preferably 50 to 150 mmol/l.

The concentration of the at least one phospholipase in the buffer solution is according to a preferred embodiment in the range of from 0.01 to 500 U/ml, more preferably in the range of from 0.05 to 100 U/ml, more preferably in the range of from 0.1 to 50 U/ml and most preferably in the range of from 0.5 to 30 U/ml. A range of from 0.3 to 30 U/ml is also preferred.

The at least one phospholipase is preferably immobilized on the surface of the carrier by non-covalent bonds. It is, however, also possible to fix the at least one phospholipase on the surface of the carrier via covalent bonds. For this, the carrier and the phospholipase are reacted with a coupling agent, which has at least two reactive groups, so that one of the groups can react with for example hydroxyl groups on the surface of the carrier and the other group can react with a suitable group of the enzyme, such as a hydroxyl, an amino or a thiol group. Examples of coupling agents are silanization reagents, polycarbonates, polyaldehydes, polyepoxides, polyazyl azides, polyisocyanates and polyazlactones. In the context of the present invention the coupling agent is preferably selected from the group consisting of silanes, polyaldehydes and polyepoxides.

For better coupling of the phospholipase used onto the carrier surface, additionally a so-called spacer can be bound to the coupling agent. Non-limiting examples of spacer molecules are glutaraldehyde, polyethylene glycol diamine, polyethylene-imine, dextran or polyethers.

It is also possible to crosslink the at least one phospholipase by corresponding at least bifunctional molecules, so as to increase the molecular weight of the enzyme and thus make it less soluble in the reaction medium. This measure can be employed when the enzyme is physically adsorbed on the carrier. Usual bifunctional molecules, which are known by a person skilled in the art to be suitable for the purpose according to the invention, can be used for the crosslinking.

The at least one phospholipase is preferably bound to the carrier via a non-covalent bond. Through the non-covalent bonding of the enzyme on the carrier, there is less disturbance of the structure of the enzyme, so that the immobilization does not excessively affect the activity of the enzyme.

The amount of the enzyme that is immobilized on the carrier is preferably 0.01 to 10 U per mg (carrier), especially preferably 0.05 to 5 U per mg, more preferably 0.1 to 3 U per mg.

To prevent premature deactivation of the enzyme, the contacting is preferably carried out at a temperature in the range of from 0 to 37° C., especially preferably in the range of from 10 to 35° C., more preferably from 15 to 30° C. and most preferably from 18 to 25° C.

Enzyme and carrier can be brought in contact in any manner. Thus, the carrier can be suspended in a solution of the enzyme.

However, it is also possible to spray the solution of the enzyme onto the carrier, for example while the latter is moving.

The time taken to immobilize the enzyme depends on the carrier used and on the enzyme used. Preferably the contacting is carried out for a period in the range of from 1 minute to 48 hours, more preferably 5 minutes to 24 hours, more preferably of from 10 minutes to 12 hours, also preferably of from 12 minutes to 3 hours and most preferably of from 15 minutes to 1 hour.

In a preferred embodiment the reaction medium is separated from the solid phospholipase-carrier complex. This can take place by the usual methods, for example by filtration or centrifugation.

In another preferred embodiment the contacting of the enzyme with the carrier is carried out in situ during the oil degumming. In this case the enzyme, dissolved in aqueous buffer solution, is brought in contact with the carrier and oil simultaneously. The immobilization of the enzyme takes place during the degumming of the oil in the aqueous phase.

In a preferred embodiment the method further comprises the step of:

e) optionally washing the separated phospholipase-carrier complex;

Optionally, unbound enzyme can be removed by washing. For washing, it is possible for example to use the same buffer as was used during the reaction of enzyme and inorganic carrier, but a different buffer can also be selected. If only relatively little solvent, especially water, is contained in the solid phospholipase-carrier complex, for example because the enzyme was sprayed onto the carrier, the solvent can also be evaporated. For this, for example the solvent can be distilled off, also under reduced pressure. Once again the temperature is selected as low as possible, i.e. preferably in a range of from 0 to 37° C., especially preferably in the range of from 10 to 35° C., more preferably of from 15 to 30° C. and most preferably of from 18 to 25° C., to avoid premature deactivation of the enzyme.

In order to avoid denaturation of the enzyme during immobilization or in order to provide a suitable pH for adsorption of the enzyme on the carriers that is as efficient as possible, in another preferred embodiment of the present invention the carriers are equilibrated to a suitable pH prior to contacting. For this, the carriers are preferably made into a slurry in a suitable buffer. The pH of the buffer is preferably selected in a range of from 3.0 to 9.0, preferably in a range of from 3.0 to 8.5 and more preferably in a range of from 3.5 to 8.0. The buffer is preferably selected the same as the buffer in which the enzyme is dissolved or taken up. The time for the equilibration of the carrier is preferably selected in the range of from 1 minute to 48 hours, more preferably 5 minutes to 24 hours, even more preferably of from 8 minutes to 12 hours and most preferably of from 10 minutes to 5 hours. Before immobilization, the buffer used for the equilibration can optionally be replaced with fresh buffer.

In another preferred embodiment, the present invention relates to a phospholipase-carrier complex, comprising at least one phospholipase A1, A2 and/or E and at least one carrier, wherein the carrier is selected from silicates and organic polymers and copolymers.

Basically the definitions given above also apply with respect to the phospholipase carrier complex according to the invention. The carrier can thus have all properties and compositions as were defined more precisely above, however, in an especially preferred embodiment, the carrier is selected from silicic acid, precipitated silicic acid, acid-activated layered silicate, hydrophobic silicate, partially hydrophobic silicate, divinylbenzene-crosslinked methacrylate, polyacrylate and polymethacrylate.

In another especially preferred embodiment, the carrier based on acid-activated layered silicate has a cation exchange capacity of less than 40 meq/100 g, preferably less than 30 meg/100 g, more preferably of less than 20 meq/100 g.

In another especially preferred embodiment the ratio of the at least one phospholipase to the carrier is 0.05-5 U/mg (carrier).

### Methods

The following methods of analysis were used:

BET surface/pore volume according to BJH and BET:

The surface area and the pore volume were determined with a fully automatic nitrogen porosimeter from the company Micromeritics, type ASAP 2010.

The sample is cooled under high vacuum to the temperature of liquid nitrogen. Then nitrogen is fed continuously into the sample chambers. By recording the amount of gas adsorbed as a function of the pressure, an adsorption isotherm is determined at constant temperature. During pressure equalizing, the analysis gas is removed progressively and a desorption isotherm is recorded.

For determining the specific surface and the porosity according to the BET theory, the data are evaluated according to DIN 66131.

The pore volume is also determined from the measured data using the BJH method (I. P. Barret, L. G. Joiner, P. P. Haienda, J. Am. Chem. Soc. 73, 1991, 373). In this method, capillary condensation effects are also taken into account. Pore volumes in certain ranges of volumes are determined by summation of incremental pore volumes, which are obtained from evaluation of the adsorption isotherm according to BJH. The total pore volume according to the BJH method relates to pores with a diameter of from 1.7 to 300 nm.

Particle Size Determination by Dynamic Light Scattering (Malvern)

The average particle size is determined with a “2000-Mastersizer” instrument from the company Malvern Instruments Ltd., UK, according to the manufacturer's instructions. The measurements are carried out in air with the sample chamber provided (“dry powder feeder”) and the values referred to the sample volume are determined.

Water Content.

The water content of the products at 105° C. is determined using the method DIN/ISO-787/2.

Determination of Bulk Density

A graduated cylinder cut off at the 1000 ml mark is weighed. Then the sample to be investigated is filled by means of a powder funnel in the graduated cylinder in a single operation, so that a cone of loose material forms above the end of the graduated cylinder. The cone of loose material is skimmed off using a ruler, which is passed across the opening of the graduated cylinder, and the filled graduated cylinder is weighed again. The difference corresponds to the bulk density.

Determination of Protein Concentration by the BCA Method and Micro-BCA Method

In the BCA method, bicinchoninic acid (BCA) serves as detection system. First, there is complexation of protein with Cu²⁺ ions in alkaline solution. The Cu²⁺ ions of the complex are reduced to Cu⁺ ions, which can be detected by complexation with BCA by measurement of absorption at 562 nm.

Procedure for the BCA Method

The determination was carried out with working reagents, which were obtained ready for use from the company Pierce, Ill., US.

25 μl of the enzyme/protein solution is pipetted into a well of a 96-well plate. 200 μl of working reagent is added by pipette and the mixture is homogenized. After incubation for 30 minutes at 37° C., the extinction at 550 nm is measured in the plate photometer.

Standard curve for protein determinations:

| | | |
|---|---|---|
| Standard 1 | 0.025 | mg/ml |
| Standard 2 | 0.125 | mg/ml |
| Standard 3 | 0.25 | mg/ml |
| Standard 4 | 0.5 | mg/ml |
| Standard 5 | 1.0 | mg/ml |
| Standard 6 | 2.0 | mg/ml |

| | |
|---|---|
| Blank value | buffer only |

Procedure for the Micro-BCA Method

The determination was carried out with working reagents, which were obtained ready for use from the company Pierce, Ill., US.

150 μl of the enzyme/protein solution is pipetted into a well of a 96-well plate. 150 μl of working reagent is added by pipette and the mixture is homogenized. After incubation for 120 minutes at 37° C., the extinction at 550 nm is measured in the plate photometer.

Standard carve for protein, determinations:

| | | |
|---|---|---|
| Standard 1 | 200 | μg/ml |
| Standard 2 | 40 | μg/ml |
| Standard 3 | 20 | μg/ml |
| Standard 4 | 10 | μg/ml |
| Standard 5 | 5 | μg/ml |
| Standard 6 | 2.5 | μg/ml |

| | |
|---|---|
| Blank value | buffer only |

Carriers Used

Commercially available carrier materials based on SiO₂, layered silicates and organic polymers were used in the following examples.

TABLE 1
Carrier materials used

| | |
|---|---|
| Carrier 1 | CAB-O-Sil ® M-5 (CABOT) |
| Carrier 2 | Sipernat 22 (Evonik) |
| Carrier 3 | KA granules (Süd-Chemie) |
| Carrier 4 | Lewatit ® VP OC 1600 (Lanxess) |
| Carrier 5 | Sipernat 22 granules |
| Carrier 6 | Aerosil R 972 (Evonik) |
| Carrier 7 | Aerosil R 974 (Evonik) |

The properties of the carrier materials are summarized in Table 2.

TABLE 2
Physical properties of the carrier materials

| | Carrier 1 | Carrier 2 | Carrier 3 | Carrier 4 | Carrier 6 | Carrier 7 |
|---|---|---|---|---|---|---|
| Bulk density | 43 | 220 | 522 | 650-800 | — | — |
| (g/l) (according | | | | | | |
| to manufacturer's | | | | | | |
| data) | | | | | | |
| Average particle | 13.25 | 12.07 | 608 | 550 | 3.27 | 7.72 |
| size (d₅₀) | | | | | | |
| [μm] (according to | | | | | | |
| Malvern) | | | | | | |
| BET surface | 200.29 | 167.10 | 149.2 | 80 | 103 | 171 |
| (m²/g) | | | | | | |
| Cumulative pore | 0.48 | 1.15 | 0.4 | 0.45 | 0.27 | 0.5 |
| volume (BJH) for | | | | | | |
| pore diameter | | | | | | |
| 1.7-300 nm (cm³/g | | | | | | |
| Average pore | 11.8 | 32.5 | 10.5 | 25.3 | 11.7 | 12.3 |
| diameter | | | | | | |
| (BJH) (nm) | | | | | | |

### BRIEF DESCRIPTION OF THE DRAWINGS

The invention is explained in more detail below on the basis of figures and examples. It is emphasized that the examples and figures are only of an illustrative nature and illustrate especially preferred embodiments of the present invention. Neither examples nor figures limit the scope of the present invention.

The figures show

FIG. 1 particle size distribution of the product Lanxess VPOC 1600 measured with a Mastersizer 2000 Vers. 5.40 (serial number MAL 1015917) from the company Malvern Instruments Ltd., UK, in air

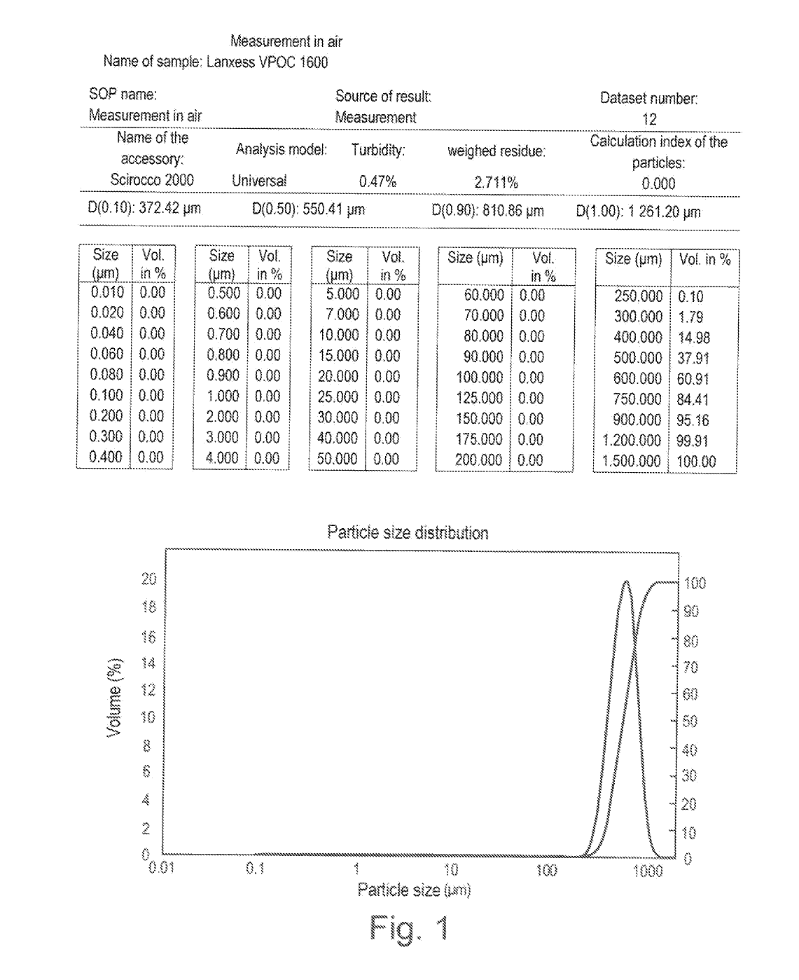

FIG. 2 particle size distribution of the product EXM 1907 measured with a Mastersizer 2900 vers. 5.40 (serial number MAL 1015917) from the company Malvern Instruments Ltd., UK, in air

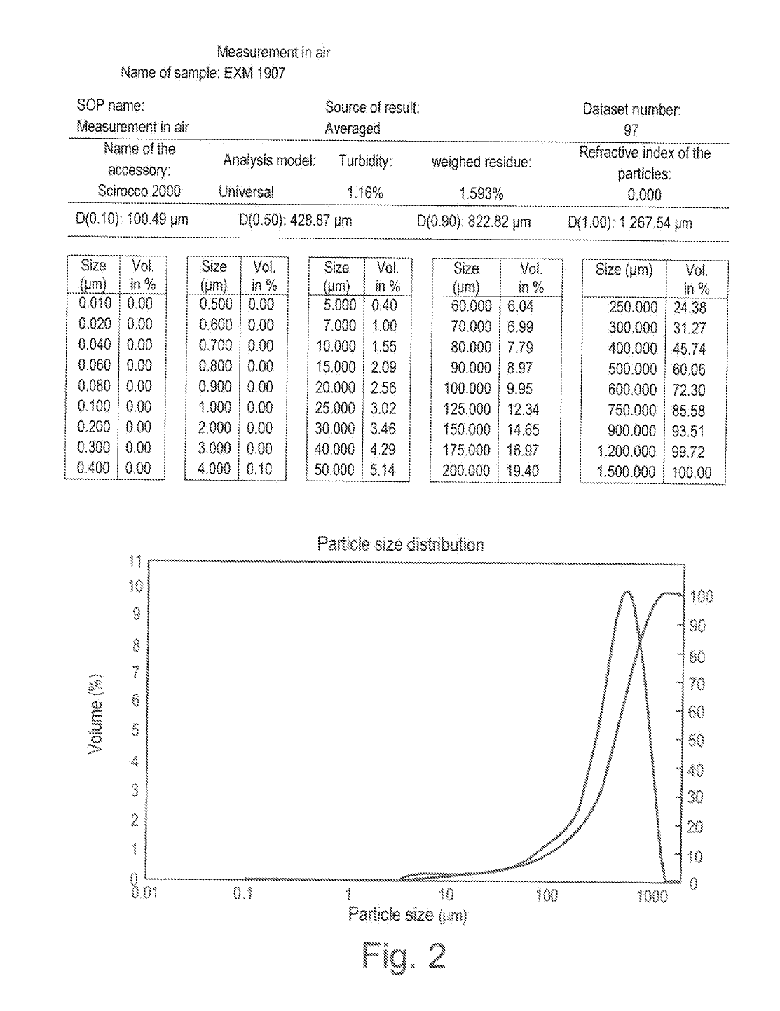

FIG. 3 particle size distribution of the product Cap-O-Sil measured with a Mastersizer 2000 Vers. 5.40 (serial number MAL 1015917) from the company Malvern Instruments Ltd., UK, in air

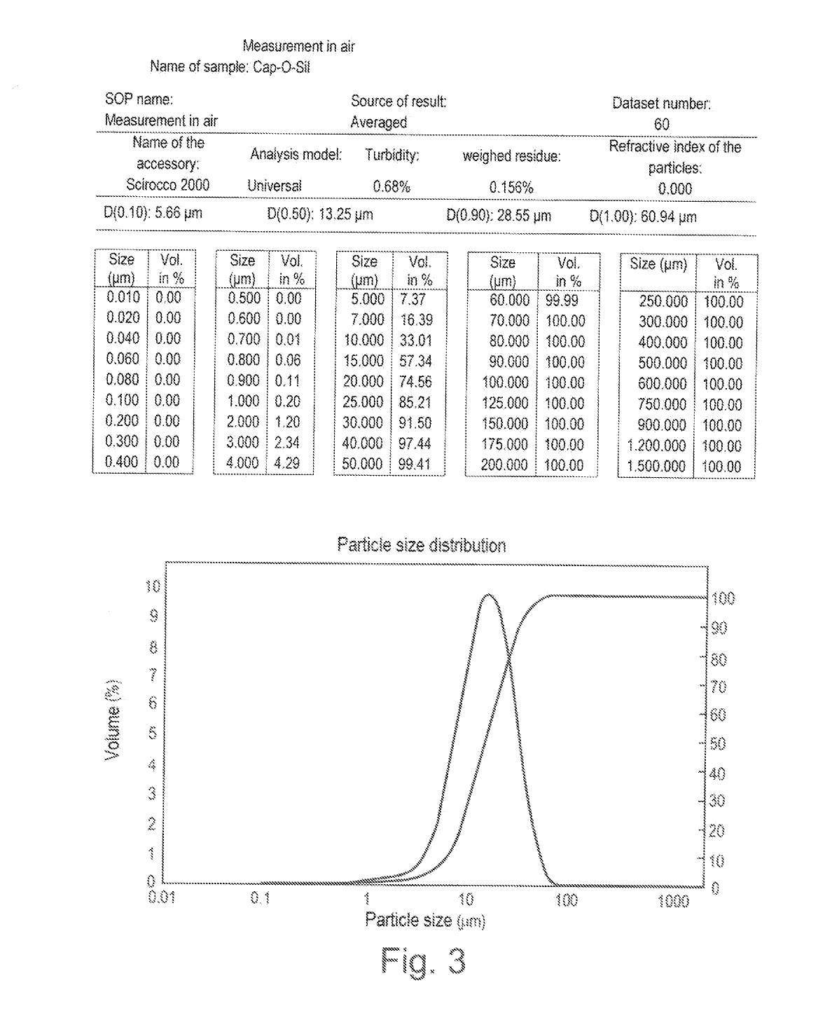

FIG. 4 particle size distribution of the product Sipernat 22 (powder) measured with a Mastersizer 2000 Vers. 5.40 (serial number MAL 1015917) from the company Malvern Instruments Ltd., UK, in air.

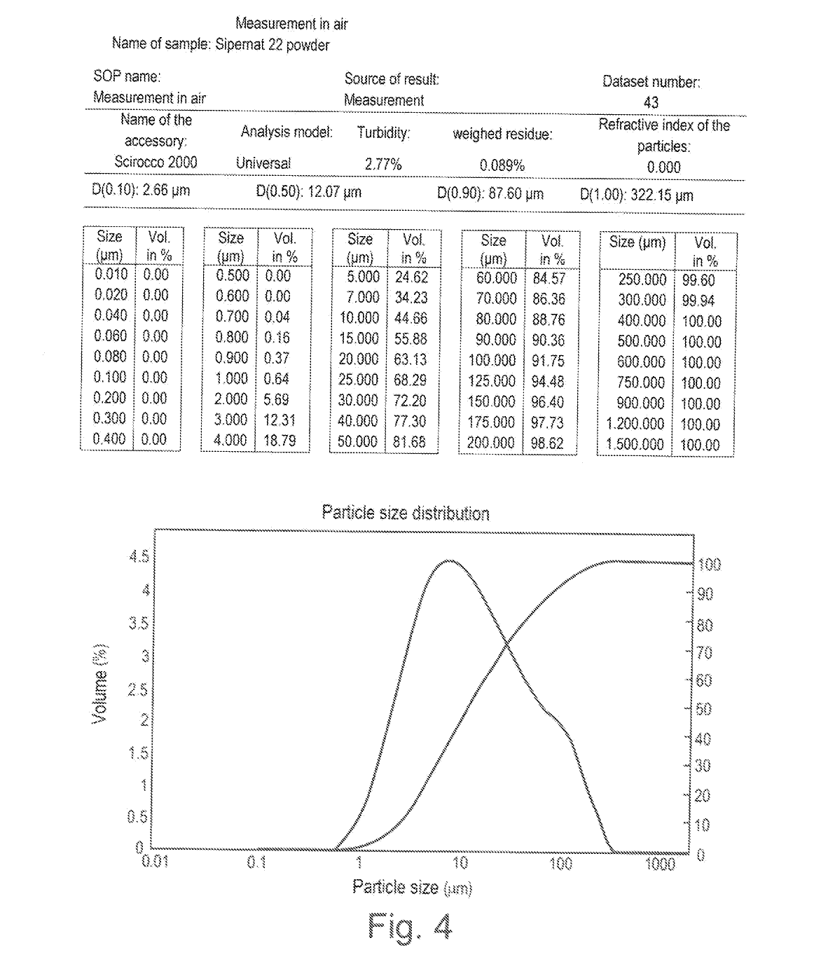

### DESCRIPTION OF THE PREFERRED EMBODIMENTS OF THE INVENTION

### Example 1

### Production of Carriers Based on Precipitated Silicic Acid

Granules were produced based on precipitated silicic acid. For this, precipitated silicic acid (Sipernat® 22, Evonik Degussa, Hanau, DE) was granulated with water. The carrier was put in an Eirich intensive mixer R=2E (company Eirich, Hartheim, DE) and water was added through a funnel. The lowest setting was selected for the rotary speed of the pan and the maximum rotary speed for the rotor. The wet granules were first dried at 70° C. and after drying, the granules were in each case sintered for one hour at 600° C. The formulation used for production of the granules is presented in Table 3.

TABLE 3
Formulations for production of granules
based on precipitated silicic acid

| | Carrier 5 |
|---|---|
| Initial weight Sipernat ® 22 [g] | 300 |
| Water [g] | 506 |

The properties of the granules obtained are shown in Table 4.

TABLE 4
Physical properties of the granules

| | Carrier 5 |
|---|---|
| Bulk density [g/l] | 343 |
| pH (10 wt % in water) | 6.1 |
| Water content (wt %) | 2.0 |
| BET surface (m²/g) | 157 |
| Cumulative pore volume according to BJH | 1.12 |
| for pores of 1.7-300 nm (cm³/g) | |
| Average pore diameter according to BJH (nm) | 28.6 |

### Example 2

### Supporting of Phospholipase A1 and Subsequent Oil Degumming

A phospholipase A1 (Lecitase™ Ultra) from Thermomyces lanuginosus (Sigma-Aldrich GmbH, Taufkirchen, DE) was used for adsorption on the carriers.

Determination of Enzyme Bonding Capacity in the Static System

Preparation of the Carriers

In each case 100 mg of the carriers is equilibrated with 2.2 ml of 50 mMol acetate buffer (pH 4.5). The carrier suspended in the buffer is shaken for 10 minutes at 20 rpm in an overhead mixer. The suspension is then centrifuged at 321.9 g and 25° C. for 10 minutes and the supernatant is discarded.

Preparation of the Enzymes

A stock solution of phospholipase A1 with a concentration of 50 U/ml is prepared in 50 mMol acetate buffer (pH 4.5).

Investigation of Enzyme Adsorption

2.2 ml of the enzyme stock solution is added in each case to 100 mg of the equilibrated carrier and the suspension is incubated for 10-90 min at room temperature and 20 rpm in the overhead shaker. Then the solids are centrifuged off at 3219 g (25° C.) for 10 min and the protein content in the supernatant is determined by the BCA or MicroBCA method. Then the solids are again suspended three times in 2.2 ml of the corresponding buffer, mixed for 10 min at room temperature and 20 rpm in the overhead shaker and centrifuged off again at 3219 g. The protein content of the wash water is also determined. The amount of enzyme adsorbed on the carrier is calculated from the difference between the amount of enzyme used and the total of the amount of enzyme measured in the supernatant and in the wash water. After the supernatant has been removed completely, 0.9 ml of 50 mMol acetate buffer (pH 4.5) and 0.1 ml of CaCl₂ (50 mM) are added to the carrier loaded with the enzyme and the suspension is used for crude oil degumming.

Blank Value of Enzyme Adsorption

For the blank value, pure acetate buffer is used instead of the enzyme solution. The blank value is treated as in the aforementioned procedure.

The amount of hound phospholipase A1 on the various carrier materials is shown in Table 5. In each case the total amount of enzyme used was bound to the carriers.

TABLE 5
Amount of phospholipase A1 bound on different carriers

| | Carrier 1 | Carrier 2 | Carrier 3 | Carrier 4 |
|---|---|---|---|---|
| bound [U] | 110 | 110 | 110 | 110 |
| bound [U/g] | 1100 | 1100 | 1100 | 1100 |
| bound [%] | 100 | 100 | 100 | 100 |

Degumming with Crude Oil

i) Measured Value of Supported Phospholipase A1

9 ml of soya oil is added to the supported enzyme. The suspension is then incubated for 16 h at 37° C. and 40 rpm in the overhead shaker. Then the samples are centrifuged at 3219 g (25° C.) for 10 min and the supernatant is removed from the carrier completely. The oil supernatant is used for phosphorus analysis. Phosphorus is determined by ICP according to DEV E-22. Buffer/oil are added to the supported enzyme again and the procedure described above is repeated. This procedure is repeated at least three times.

ii) Blank Value of Supported Phospholipase A1

The carrier without immobilized enzyme serves as blank value. The degumming with crude oil proceeds similarly to as under i.

iii) Measured Value of Unsupported Phospholipase A1

The same concentration of unsupported phospholipase A1 was used as in the test under i. 0.1 ml of CaCl₂ solution (50 mM) and 9 ml of soya oil are added to 0.9 ml (110 U) of PLA1 in 50 mM acetate buffer (pH 4.5). The suspension is then incubated for 16 h at 37° C. and 40 rpm in the overhead shaker. Then the samples are centrifuged at 3219 g (25° C.) for 10 min. The oil supernatant is used for phosphorus analysis.

iv) Blank Value of Unsupported Phospholipase A1

In the case of the blank value of unsupported phospholipase, the test is carried out as under iii, with buffer, without enzyme solution.

The results for the degumming of soya oil with immobilized phospholipase A1 are presented in Table 6. In each step, the oil was incubated with the enzyme for 16 h at 37° C.

TABLE 6
Phosphorus values before and after degumming of soya oil with immobilized and non-immobilized
PLA1. Abbreviations: BV = blank value; MV = measured value; Carr. = Carrier

| | | | | BV | MV | BV | MV | BV | MV | BV | MV |
|---|---|---|---|---|---|---|---|---|---|---|---|
| | Crude | BV | MV | Carr. | Carr. | Carr. | Carr. | Carr. | Carr. | Carr. | Carr. |
| | oil | Enzyme | Enzyme | 1 | 1 | 2 | 2 | 3 | 3 | 4 | 4 |
| Soya oil | 275 | | | | | | | | | | |
| without carrier | | 65.5 | 0.5 | | | | | | | | |
| 1st degumming | | | | 65.6 | 0.5 | 56.8 | 0.5 | 61.3 | 0.5 | 65.6 | 2.6 |
| 1st recycling | | | | 56 | 2.6 | 46.7 | 0.5 | 50.3 | 0.5 | 54 | 2.1 |
| 2nd recycling | | | | 53 | 2 | 47 | 2.7 | 60 | 1.4 | 63 | 2.9 |
| 3rd recycling | | | | 50 | 3.9 | 43 | 1.7 | 47 | 1.5 | 57 | 0.5 |
| 4th recycling | | | | 54.8 | 0.5 | 40 | 3.5 | 53 | 1.1 | 55 | 0.5 |
| 5th recycling | | | | 59 | 0.5 | 42 | 3.9 | 48 | 1.9 | 56 | 0.5 |

### Example 3

### Supporting of Phospholipase A2 and Subsequent Oil Degumming

A phospholipase A2 from pig pancreas was used (Sigma-Aldrich GrabH, Taufkirchen, DE) for adsorption on the carriers.

For phospholipase A2, a 50 mMol acetate buffer (pH 4) was used for immobilization and for all further steps. Otherwise the test was carried out similarly to the test in example 2.

The amount of phospholipase A2 bound to the various carrier materials is shown in Table 8. In each case the total amount of enzyme used was bound to the carriers.

TABLE 8
Amount of phospholipase A2 bound to different carriers

| | Carrier 1 | Carrier 2 | Carrier 3 | Carrier 4 | Carrier 5 |
|---|---|---|---|---|---|
| bound [U] | 110 | 110 | 110 | 110 | 110 |
| bound [U/g] | 1100 | 1100 | 1100 | 1100 | 1100 |
| bound [%] | 100 | 100 | 100 | 100 | 100 |

Results of Degumming

The results for degumming of soya oil and rape oil with immobilized phospholipase A2 are presented in Tables 9 and 10. In each step, the oil was incubated with the enzyme for 16 h at 37° C.

TABLE 9
Phosphorus values before and after degumming of soya oil with immobilized and non-immobilized
PLA2. Abbreviations: BV = blank value; MV = measured value; carr. = carrier

| | | | | BV | MV | BV | MV | BV | MV | BV | MV | BV. | MV. |
|---|---|---|---|---|---|---|---|---|---|---|---|---|---|
| | Crude | BV | MV | carr. | carr. | carr. | carr. | carr. | carr. | carr. | carr. | carr. | carr. |
| | oil | Enzyme | Enzyme | 1 | 1 | 2 | 2 | 3 | 3 | 4 | 4 | 5 | 5 |
| Soya oil | 275 | | | | | | | | | | | | |
| without carrier | | 58 | 1.8 | | | | | | | | | | |
| 1st degumming | | | | 65.5 | 1.1 | 53 | 1.5 | 57 | 1.8 | 62 | 0.5 | 46 | 1.3 |
| 1st recycling | | | | 50 | 2.5 | 45 | 1.8 | 50 | 2.3 | 60 | 3.1 | 42 | 1.8 |
| 2nd recycling | | | | 49 | 2.1 | 60 | 2.2 | 63 | 2.4 | 54 | 0.5 | 65 | 2.4 |
| 3rd recycling | | | | 52 | 1.1 | 67 | 2.2 | 63 | 2.4 | 48 | 1.2 | 54 | 1.7 |
| 4th recycling | | | | 54.1 | 0.5 | 58 | 2.1 | 58 | 2.4 | 50 | 0.5 | 51 | 1.7 |
| 5th recycling | | | | 64.1 | 0.5 | 63 | 0.5 | 58.3 | 0.8 | 50 | 0.5 | 57.7 | 0.8 |
| 6th recycling | | | | 58.7 | 0.5 | 62.7 | 0.5 | 55.7 | 0.9 | 61 | 0.5 | 56 | 0.5 |
| 7th recycling | | | | 70.3 | 1.2 | 64 | 1.8 | 57 | 2.1 | 57 | 1 | 60 | 1.9 |
| 8th recycling | | | | 62.5 | 0.9 | 55 | 1.8 | 46 | 1.7 | 55 | 1.4 | 52 | 1.7 |
| 9th recycling | | | | 60.2 | 0.7 | 62.1 | 0.5 | 51.7 | 0.5 | 55 | 0.6 | 44 | 1.9 |
| 10th recycling | | | | 61.7 | 0.5 | 59.8 | 0.5 | 55.1 | 2.1 | 57 | 0.5 | 43 | 2 |

TABLE 10
Phosphorus values before and after degumming rape oil with immobilized and
non-immobilized PLA2. Abbreviations: BV = blank value; MV = measured value

| | | | | BV | MV | BV | MV |
|---|---|---|---|---|---|---|---|
| | Crude | BV | MV | Carrier | Carrier | Carrier | Carrier |
| | oil | Enzyme | Enzyme | 2 | 2 | 3 | 3 |
| Rape oil | 243 | | | | | | |
| without carrier | | 144 | 30.9 | | | | |
| 1st degumming | | | | 145 | 0.5 | 151 | 4 |
| 1st recycling | | | | 140 | 0.5 | 146 | 1 |
| 2nd recycling | | | | 140 | 0.5 | 143 | 0.7 |
| 3rd recycling | | | | 144 | 0.5 | 146 | 0.7 |
| 4th recycling | | | | 145 | 0.5 | 143 | 1.3 |
| 5th recycling | | | | 110 | 2.8 | 83 | 3.6 |
| 6th recycling | | | | 80 | 2.4 | 120 | 3.2 |
| 7th recycling | | | | 118 | 3.4 | 129 | 2.4 |
| 8th recycling | | | | 136 | 2.2 | 143 | 1.9 |
| 9th recycling | | | | 135 | 3.8 | 134 | 1.9 |

### Example 4

### Decrease in Concentration of Carrier 2/ Immobilized Phospholipase A2

A phospholipase A2 from pig pancreas (Sigma-Aldrich GmbH, Taufkirchen, DE) was used for adsorption on the carriers.

Preparation of Carrier 2

In each case 50 mg and 25 mg of carrier 2 are equilibrated with 2.2 ml of 50 mMol acetate buffer (pH 4). The carrier suspended in the buffer is shaken for 10 minutes at 20 rpm in an overhead mixer. The suspension is then centrifuged at 3219 g and 25° C. for 10 minutes and the supernatant is discarded.

Preparation of the Enzymes

For the examples, a stock solution of phospholipase A2 with a concentration of 50 U/ml is prepared in 50 mMol of acetate buffer (pH 4).

Investigation of Enzyme Adsorption

1.1 ml of enzyme stock solution is added in each case to 50 mg of equilibrated carrier 2, 0.55 ml of enzyme stock solution is added in each case to 25 mg of equilibrated carrier 2 and the suspension is incubated for 90 min at 25° C. and 20 rpm in the overhead shaker. Then the solids are centrifuged off at 3219 g (25° C.) for 10 min and the protein content in the supernatant is determined by the ECA method. Then the solids are suspended again three times in 2.2 ml of the corresponding buffer, mixed for 10 min at room temperature and 20 rpm in the overhead shaker and centrifuged off again at 3219 g. The protein content of the wash water is also determined. The amount of enzyme adsorbed on the carrier was calculated from the difference between amount of enzyme used and the total amount of enzyme measured in the supernatant and in the wash water. All tests are carried out as triple determination. After the supernatant has been removed completely, 0.9 ml of 50 mMol acetate buffer (pH 4) and 0.1 ml of CaCl₂ (50 mM) are added to the carrier loaded with the enzyme and the suspension is used for crude oil degumming.

Blank Value of Enzyme Adsorption

For the blank value, pure acetate buffer is used instead of the enzyme solution. The blank value is treated as in the procedure described above.

The amount of phospholipase A2 bound to the different carrier materials is shown in Table 11. The total amount of phospholipase A2 used has bound to the carriers.

TABLE 11
Amount of phospholipase A2 bound to different
amounts of carrier 2 at pH 4

| | Carrier 2 (50 mg) | Carrier 2 (25 mg) |
|---|---|---|
| bound [U] | 55 | 27.5 |
| bound [U/g] | 1100 | 1100 |
| bound [%] | 100 | 100 |

Degumming with Soya Oil

The degumming with soya oil takes place similarly to the description in example 2.

The results for the degumming of soya oil with immobilized phospholipase A2 are presented in Table 12. In each step, the oil was incubated with the enzyme for 16 h at 37° C.

TABLE 12
Phosphorus values before and after degumming of soya oil with immobilized and
non-immobilized PLA2. Abbreviations: BV = blank value; MV = measured value

| | | | | BV | MV | | BV | MV |
|---|---|---|---|---|---|---|---|---|
| | Crude | BV | MV | Carrier | Carrier | Mv | Carrier | Carrier |
| | oil | Enzyme | Enzyme | 2 | 2 | Enzyme | 2 | 2 |
| Soya oil | 248 | | (55 U) | 50 mg | 55 U/ | (27.5 U) | 25 mg | 27.5 U/ |
| | | | | | 50 mg | | | 25 mg |
| without carrier | | 50 | 3.8 | | | 8.9 | | |
| (110 U) | | | | | | | | |
| 1st degumming | | | | 56.5 | 0.5 | | 58.8 | 0.5 |
| 1st recycling | | | | 54.3 | 0.5 | | 54.8 | 0.5 |
| 2nd recycling | | | | 54.5 | 0.5 | | 55.5 | 0.5 |
| 3rd recycling | | | | 57.6 | 0.5 | | 55.2 | 0.5 |
| 4th recycling | | | | 53.1 | 0.5 | | 53.4 | 0.5 |
| 5th recycling | | | | 60 | 0.5 | | 55.8 | 0.5 |
| 6th recycling | | | | 46 | 0.8 | | 48 | 0.8 |
| 7th recycling | | | | 32 | 1.7 | | 48 | 1.8 |
| 8th recycling | | | | 46 | 2 | | 50 | 2.4 |
| 9th recycling | | | | 45 | 2.8 | | 46 | 2.3 |
| 10th recycling | | | | 49 | 2.2 | | 57 | 3.7 |

### Example 5

### Supporting of phospholipase A1 and A2 on Hydrophobia/Partially Hydrophobic Carrier and Subsequent Oil Degumming

A phospholipase A1 and A2 was used for the oil degumming.

Preparation of the Carriers

In each case 15 mg of the carriers is equilibrated with 5 ml of 500 mMol citrate buffer (pH 5) for 10 min. The suspension is then centrifuged at 3219 g and 25° C. for 10 minutes and the supernatant is discarded.

Immobilization

15 units of PLA 1 or PLA 2 in 500 mMol citrate buffer are added in each case to 15 mg of the equilibrated carrier and the suspension is incubated for 10 min at room temperature and 20 rpm in the overhead shaker. Then the solids are centrifuged off at 3219 g (25° C.) for 10 min. 0.25 ml of 500 mMol citrate buffer (pH 5) is added to the carrier loaded with the enzyme and the suspension is used for crude oil degumming.

Blank Value

For the blank value, pure citrate buffer is used instead of the enzyme solution. The blank value is treated as in the aforementioned procedure.

Degumming with Crude Oil

i) Measured Value of Supported Phospholipase A1 or A2

4.75 ml of soya oil is added to the supported enzyme. The suspension is then incubated for 4 h at 48° C. (PLA 1) or for 2 h at 55° C. (PLA 2) and 40 rpm in the overhead shaker. Then the samples are centrifuged at 3219 g (25° C.) for 10 min and the supernatant is removed from the carrier completely. The oil supernatant is used for phosphorus analysis. Phosphorus was determined by ICP according to DEV E-22. Buffer/oil (ratio 5%/95%) is added to the supported enzyme again and the operation described above is repeated. This procedure is repeated at least three times.

ii) Blank Value of Supported Phospholipase A1 or A2

The carrier without immobilized enzyme serves as blank value. The degumming with crude oil proceeds as under i.

iii) Measured Value of Unsupported Phospholipase

The same concentration of unsupported phospholipase A1 or A2 was used as in the test under i. 5 ml of soya oil is added to 0.25 ml (15 U) PLA1 or A2 in 500 mM citrate buffer (pH 5). The suspension is then incubated for 4 h at 48° C. (A1) or 55° C. (A2) and 4.0 rpm in the overhead shaker. Then the samples are centrifuged at 3219 g (25° C.) for 10 min. The oil supernatant is used for phosphorus analysis.

iv) Blank Value of Unsupported Phospholipase

For the blank value of unsupported phospholipase, the test is carried out with buffer, without enzyme solution, as under iii.

TABLE 13
Phosphorus values before and after degumming of soya oil with immobilized and non-
immobilized PLA1 and PLA2. Abbreviations: BV = blank value; MV = measured value

| | | | | BV | MV | BV | MV |
|---|---|---|---|---|---|---|---|
| | Crude | BV | MV | Carrier | Carrier | Carrier | Carrier |
| | oil | Enzyme | Enzyme | 6 | 6 | 7 | 7 |
| Soya oil | 815 | | | | | | |
| without carrier | | 75.6 | 70 | | | | |
| (PLA 1) | | | | | | | |
| 1st degumming (PLA 1) | | | | 75.5 | 1.49 | 76 | 1.3 |
| 1st recycling (PLA 1) | | | | 75.5 | 0.1 | 76 | 0.1 |
| 2nd recycling (PLA 1) | | | | 75.5 | 0.1 | 76 | 0.1 |
| 3rd recycling (PLA 1) | | | | 75.5 | 0.1 | 76 | 0.1 |
| without carrier | | 75.6 | 75.5 | | 0.5 | | |
| (PLA 2) | | | | | | | |
| 1st degumming (PLA 2) | | | | 75.5 | 0.1 | 76 | 0.1 |
| 1st recycling (PLA 2) | | | | 75.5 | 0.1 | 76 | 0.1 |
| 2nd recycling (PLA 2) | | | | 75.5 | 0.1 | 76 | 0.1 |
| 3rd recycling (PLA 2) | | | | 75.5 | 0.1 | 76 | 0.1 |

TABLE 14
Calcium values before and after degumming of soya oil with immobilized and non-
immobilized PLA1 and PLA2. Abbreviations: BV = blank value; MV = measured value

| | | | | BV | MV | BV | MV |
|---|---|---|---|---|---|---|---|
| | Crude | BV | MV | Carrier | Carrier | Carrier | Carrier |
| | oil | Enzyme | Enzyme | 6 | 6 | 7 | 7 |
| Soya oil | 98 | | | | | | |
| without carrier | | 56.1 | 9.6 | | | | |
| (PLA 1) | | | | | | | |
| 1st degumming (PLA 1) | | | | 55 | 0.1 | 56 | 0.1 |
| 1st recycling (PLA 1) | | | | 55 | 0.1 | 56 | 0.1 |
| 2nd recycling (PLA 1) | | | | 55 | 0.1 | 56 | 0.1 |
| 3rd recycling (PLA 1) | | | | 55 | 0.4 | 56 | 0.4 |
| without carrier | | 56.1 | 56 | | | | |
| (PLA 2) | | | | | | | |
| 1st degumming (PLA 2) | | | | 55 | 0.1 | 56 | 0.1 |
| 1st recycling (PLA 2) | | | | 55 | 0.1 | 56 | 0.1 |
| 2nd recycling (PLA 2) | | | | 55 | 0.3 | 56 | 0.5 |
| 3rd recycling (PLA 2) | | | | 55 | 0.2 | 56 | 0.3 |

TABLE 15
Magnesium values before and after degumming of soya oil with immobilized and non-
immobilized PLA1 and PLA2. Abbreviations: BV = blank value; MV = measured value

| | | | | BV | MV | BV | MV |
|---|---|---|---|---|---|---|---|
| | Crude | BV | MV | Carrier | Carrier | Carrier | Carrier |
| | oil | Enzyme | Enzyme | 6 | 6 | 7 | 7 |
| Soya oil | 97 | | | | | | |
| without carrier | | 29.8 | 16.8 | | | | |
| (PLA 1) | | | | | | | |
| 1st degumming (PLA 1) | | | | 14.3 | 0.1 | 12.5 | 0.2 |
| 1st recycling (PLA 1) | | | | 14.3 | 0.1 | 12.5 | 0.2 |
| 2nd recycling (PLA 1) | | | | 14.3 | 0.1 | 12.5 | 0.1 |
| 3rd recycling (PLA 1) | | | | 14.3 | 0.15 | 12.5 | 0.2 |
| without carrier | | 17.9 | 17.5 | | | | |
| (PLA 2) | | | | | | | |
| 1st degumming (PLA 2) | | | | 16.3 | 0.1 | 12 | 0.2 |
| 1st recycling (PLA 2) | | | | 16.3 | 0.1 | 12 | 0.1 |
| 2nd recycling (PLA 2) | | | | 16.3 | 0.2 | 12 | 0.2 |
| 3rd recycling (PLA 2) | | | | 16.3 | 0.1 | 12 | 0.2 |

### Example 6

### Supporting of Phospholipase A1 and Subsequent Oil Degumming with Previously Degummed Oil

A phospholipase A1 was used for the adsorption on the carriers.

Preparation of the Carriers

In each ease 15 mg of the carriers is equilibrated with 5 ml of 50 mMol citrate buffer (pH 5) for 10 min. The suspension is then centrifuged at 3219 g and 25° C. for 10 minutes and the supernatant is discarded.

Immobilization

15 units of PLA 1 in 50 mMol citrate buffer are added in each case to 15 mg of the equilibrated carrier and the suspension is incubated for 10 min at room temperature and 20 rpm in the overhead shaker. Then the solids are centrifuged off at 3219 g (25° C.) for 10 min. 0.15 ml of 50 mMol citrate buffer (pH 5) is added to the carrier loaded with the enzyme and the suspension is used for crude oil degumming.

Blank Value

For the blank value, pure citrate buffer is used instead of the enzyme solution. The blank value is treated similarly to the aforementioned procedure.

Preliminary Degumming of Soya Oil with Citric Acid

100 ml of soya oil is heated to 40° C. and 5% of a 10% citric acid is stirred for 15 min at 40° C. Then the suspension is centrifuged off for 10 min at 3219 g and the soya oil in the supernatant is used further for enzymatic degumming.

Degumming with Soya Oil Previously Degummed with Acid

i) Measured Value of Supported Phospholipase A1

4.85 ml of previously degummed soya oil is added to the supported enzyme. The suspension is then incubated for 2 h at 48° C. and 40 rpm in the overhead shaker. Then the samples are centrifuged at 3219 g (25° C) for 10 min and the supernatant is removed from the carrier completely. The oil supernatant is used for phosphorus analysis. Buffer/oil (ratio 3%/97%) is added again to the supported enzyme and the procedure described above is repeated. This procedure is repeated at least three times.

ii) Blank Value of Supported Phospholipase A1

The carrier without immobilized enzyme serves as blank value. The degumming with crude oil proceeds as under i.

iii) Measured Value of Unsupported Phospholipase A1

The same concentration of unsupported phospholipase A1 was used as in the test under i. (pH 5) 4.85 ml of soya oil is added to 0.15 ml (15 U) PLA1 in 50 mM citrate buffer. The suspension is then incubated for 2 h at 48° C. and 40 rpm in the overhead shaker. Then the samples are centrifuged at 3219 g (25° C.) for 10 min. The oil supernatant is used for phosphorus analysis.

iv) Blank Value of Unsupported Phospholipase A1

For the blank value of unsupported phospholipase, the test is carried out with buffer, without enzyme solution, as under iii.

TABLE 16
Phosphorus values before and after degumming of soya oil with immobilized and
non-immobilized PLA 1. Abbreviations: BV = blank value; MV = measured value

| | Previously | | | BV | MV | BV | MV |
|---|---|---|---|---|---|---|---|
| | degummed | BV | MV | Carrier | Carrier | Carrier | Carrier |
| | oil | Enzyme | Enzyme | 2 | 2 | 4 | 4 |
| Soya oil | 35.5 | | | | | | |
| without carrier | | 35 | 1.1 | | | | |
| 1st degumming | | | | 33.5 | 0.5 | 36 | 0.5 |
| 1st recycling | | | | 33.5 | 0.6 | 36 | 0.5 |
| 2nd recycling | | | | 33.5 | 0.6 | 36 | 0.7 |
| 3rd recycling | | | | 33.5 | 0.7 | 36 | 0.7 |
| 4th recycling | | | | 33.5 | 0.6 | 36 | 1.0 |
| 5th recycling | | | | 33.5 | 0.7 | 36 | 3.0 |
| 6th recycling | | | | 33.5 | 0.8 | 36 | 3.0 |

TABLE 17
Calcium values before and after degumming of soya oil with immobilized and
non-immobilized PLA 1. Abbreviations: BV = blank value; MV measured value

| Previously | | | BV | MV | BV | MV |
|---|---|---|---|---|---|---|
| degummed | BV | MV | Carrier | Carrier | Carrier | Carrier |
| oil | Enzyme | Enzyme | 2 | 2 | 4 | 4 |

| | | | | | | |
|---|---|---|---|---|---|---|
| Soya oil | | | | | | |
| without carrier | 5.8 | 0.5 | | | | |
| 1st degumming | | | 7.6 | 0.5 | 6.2 | 0.5 |
| 1st recycling | | | 7.6 | 0.5 | 6.2 | 0.5 |
| 2nd recycling | | | 7.6 | 0.5 | 6.2 | 0.5 |
| 3rd recycling | | | 7.6 | 0.5 | 6.2 | 0.5 |
| 4th recycling | | | 7.6 | 0.5 | 6.2 | 0.5 |
| 5th recycling | | | 7.6 | 0.5 | 6.2 | 1.1 |

TABLE 18
Magnesium values before and after degumming of soya oil with immobilized and
non-immobilized PLA 1. Abbreviations: BV = blank value; MV = measured value

| Previously | | | BV | MV | BV | MV |
|---|---|---|---|---|---|---|
| degummed | BV | MV | Carrier | Carrier | Carrier | Carrier |
| oil | Enzyme | Enzyme | 2 | 2 | 4 | 4 |

| | | | | | | |
|---|---|---|---|---|---|---|
| Soya oil | | | | | | |
| without carrier | 1.2 | 0.5 | | | | |
| 1st degumming | | | 3.0 | 0.5 | 3.6 | 0.5 |
| 1st recycling | | | 3.0 | 0.5 | 3.6 | 0.5 |
| 2nd recycling | | | 3.0 | 0.5 | 3.6 | 0.5 |
| 3rd recycling | | | 3.0 | 0.5 | 3.6 | 0.5 |
| 4th recycling | | | 3.0 | 0.5 | 3.6 | 0.5 |
| 5th recycling | | | 3.0 | 0.5 | 3.6 | 0.5 |

### Example 7

### Supporting of Phospholipase A1 and Subsequent Oil Degumming with Previously Degummed and Partially Neutralized Oil

Supporting of the Enzyme

1.68 g of carrier is incubated with 10 ml of citrate buffer (100 mMol) for 10 min at room temperature in the overhead shaker at 40 rpm. Then 56-280 μl ml of phospholipase A1 or A2 stock solution (10000 U/ml) is added and incubated for a further 10 min. The suspension is centrifuged off for 10 min at 3219 g (25° C.).

Blank Value of Enzyme Adsorption

For the blank value, pure citrate buffer is used instead of the enzyme solution. The blank value is treated similarly to the aforementioned, procedure.

Oil Degumming

i) Measured Value of Supported Phospholipase A1

For oil degumming, 560 g of soya oil is put in a Duran glass reactor and heated to 50° C. 1.215 ml of 30% citric acid is added to the soya oil and homogenized for 1 min in the Ultrathurrax and stirred for 15 min at 50° C. at 400-600 rpm with a propeller stirrer. Then 2.7 ml of 1M NaOH is added and it is stirred for a further 5 min at 50° C. Then the supported enzyme with 15-28 ml of distilled water is added to the suspension and stirred for 180 min at 50° C. Then the suspension is centrifuged off for 10 min at 3219 g.

Phosphorus is determined by ICP according to DEV E-22. Buffer/oil (ratio 3-5%/95-97%) is again added to the supported enzyme and the procedure described above is repeated. This procedure is repeated at least three times.

ii) Blank Value of Supported Phospholipase A1

The carrier without immobilized enzyme serves as blank value. The degumming with crude oil proceeds as under i.

iii) Measured Value of Unsupported Phospholipase A1

The procedure is the same as under i. The enzyme is used in non-immobilized form. The oil supernatant is used for phosphorus analysis.

TABLE 19
Phosphorus values before and after degumming of soya
oil with immobilized and non-immobilized PLA 1. Abbreviations:
BV = blank value; MV = measured value

| | Crude | BV | MV | BV | MV |
|---|---|---|---|---|---|
| | oil | Enzyme | Enzyme | Carrier 2 | Carrier 2 |
| Soya oil | 805 | | | | |
| without | | 13.5 | 3.5 | | |
| carrier | | | | | |
| 1st | | | | 15 | 3.0 |
| degumming | | | | | |
| 1st | | | | 15 | 3.5 |
| recycling | | | | | |
| 2nd | | | | 15 | 4.0 |
| recycling | | | | | |
| 3rd | | | | 15 | 4.5 |
| recycling | | | | | |

TABLE 20
Calcium values before and after degumming of soya
oil with immobilized and non-immobilized PLA 1. Abbreviations:
BV = blank value; MV = measured value

| | | BV | MV | BV | MV |
|---|---|---|---|---|---|
| | Oil | Enzyme | Enzyme | Carrier 2 | Carrier 2 |
| Soya oil | 96 | | | | |
| without | | 9.2 | 2.1 | | |
| carrier | | | | | |
| 1st | | | | 9.8 | 1.4 |
| degumming | | | | | |
| 1st | | | | 9.8 | 2.7 |
| recycling | | | | | |
| 2nd | | | | 9.8 | 2.9 |
| recycling | | | | | |
| 3rd | | | | 9.8 | 2.8 |
| recycling | | | | | |

TABLE 21
Magnesium values before and after degumming of soya
oil with immobilized and non-immobilized PLA 1. Abbreviations:
BV = blank value; MV = measured value

| | | BV | MV | BV | MV |
|---|---|---|---|---|---|
| | Oil | Enzyme | Enzyme | Carrier 2 | Carrier 2 |
| Soya oil | 89 | | | | |
| without | | 11.8 | 1.0 | | |
| carrier | | | | | |
| 1st | | | | 10.4 | 0.4 |
| degumming | | | | | |
| 1st | | | | 10.4 | 0.8 |
| recycling | | | | | |
| 2nd | | | | 10.4 | 0.8 |
| recycling | | | | | |
| 3rd | | | | 10.4 | 0.8 |
| recycling | | | | | |

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

All publications, patents and patent applications cited in this specification are herein incorporated by reference, and for any and all purpose, as if each individual publication, patent or patent application were specifically and individually indicated to be incorporated by reference. In the case of inconsistencies, the present disclosure will prevail.

It is understood that the foregoing description is that of the preferred embodiments of the invention and that various changes and modifications may be made thereto without departing from the spirit and scope of the invention as defined in the appended claims.

What is claimed is:

1. A phospholipase-carrier complex comprising at least one phospholipase A1, A2 or B or a mixture thereof and at least one carrier for degumming crude oils.

2. The phospholipase-carrier complex according to claim 1, wherein the carrier has a pore volume according to BJH of more than 0.1 ml/g for pores with a diameter of from 1.7 to 300 nm.

3. The phospholipase-carrier complex according to claim 1, wherein the carrier has a pore diameter according to BJH of more than 2 nm.

4. The phospholipase-carrier complex according to claim 1, wherein the carrier is selected from silicates and organic polymers or copolymers.

5. The phospholipase-carrier complex according to claim 4, wherein the silicate is selected from the group consisting of natural and synthetic silicates and layered silicates or a mixture thereof.

6. The phospholipase-carrier complex according to claim 5, wherein the silicate is based on silicon dioxide.

7. The phospholipase-carrier complex according to claim 4, wherein the organic polymer or copolymer is selected from the group consisting of polyacrylate, polymethacrylate, polyethylene, polyethylene terephthalate, polytetrafluorethylene, polypropylene, polyvinyl styrene, polystyrene, styrene-divinylbenzene copolymers, polymethylmethacrylate, and polyamide or a mixture thereof.

8. The phospholipase-carrier complex according to claim 6, wherein the silicate is silicic acid, precipitated silicic acid or a mixture thereof.

9. The phospholipase-carrier complex according to claim 1, wherein the carrier is selected from the group consisting of silicic acid, precipitated silicic acid, polyacrylate, polymethacrylate and divinylbenzene-crosslinked methacrylate.

10. The phospholipase-carrier complex according to claim 9, wherein a ratio of the at least one phospholipase to the carrier is of from 0.05 to 5 U/mg (carrier).

11. A method of degumming crude oil, comprising:
a) mixing a phospholipase-carrier complex comprising at least one phospholipase A1, A2 or B or a mixture thereof and at least one carrier with a buffer solution in a ratio of 0.01%-80% (weight of phospholipase-carrier complex to volume of buffer solution);
b) contacting the buffer solution containing the phospholipase-carrier complex with a crude oil; and
c) separating the crude oil from the buffer solution containing the phospholipase-carrier complex.

12. The method according to claim 11, further comprising:
d) repeatedly contacting the buffer solution containing the phospholipase-carrier complex with a crude oil.

13. The method according to claim 11, wherein a proportion of the buffer solution containing the phospholipase-carrier complex is adjusted to a range of from 0.01 to 15 wt % relative to the crude oil.

14. The method according to claim 11, wherein a proportion of enzyme relative to the crude oil is adjusted to a range of from 0.01 to 15 units per gram of oil.

15. The method according to claim 11, wherein the crude oil is selected from the group consisting of soya oil, rape oil, palm oil, sunflower oil, canola oil, rice germ oil, peanut oil, coconut oil, pumpkin seed oil, maize germ oil, olive oil, jojoba oil, jatropha oil, walnut oil, grapeseed oil, almond oil, linseed oil, and cottonseed oil or a mixture thereof.

16. A method of producing a phospholipase-carrier complex, comprising:
a) providing at least one phospholipase A1, A2 or B or a mixture thereof in a buffer solution with a pH of from 3.0 to 8.0;
b) providing at least one carrier selected from at least one silicate and at least one organic polymer;
c) contacting the at least one phospholipase A1, A2 or B or the mixture thereof with the at least one carrier; and
d) optionally, separating the resultant phospholipase-carrier complex from the buffer solution.
